(19) United States
(12) Patent Application Publication
(10) Pub. No.: US 2026/0191775 A1
(43) Pub. Date: Jul. 9, 2026

(54) TONER, LYSOZYME BUCCAL TABLET AND PREPARATION METHOD OF LYSOZYME BUCCAL TABLET

(71) Applicant: Hangzhou Guoguang Pharmaceutical Co., Ltd., Hangzhou (CN)

(72) Inventors: Gongmin SHEN, Hangzhou (CN); Qun LIU, Hangzhou (CN); Yan JIN, Hangzhou (CN); Fengjun MO, Hangzhou (CN)

(21) Appl. No.: 19/553,570

(22) Filed: Mar. 2, 2026

Related U.S. Application Data
(62) Division of application No. 17/319,113, filed on May 13, 2021.
Application No. 17/319,113 is a continuation of application No. PCT/CN2021/077412, filed on Feb. 23, 2021.

(30) Foreign Application Priority Data
Aug. 25, 2020 (CN) 202010866046.7

(51) Int. Cl. A61K 9/00; A61K 9/20; A61K 38/47

(52) U.S. Cl. CPC A61K 9/006; A61K 9/2013; A61K 9/2018; A61K 9/205; A61K 9/2095; A61K 38/47; C12Y 302/01017

(57) ABSTRACT

The present application relates to the technical field of medicine processing, in particular to a toner, a lysozyme buccal tablet and a preparation method of the lysozyme buccal tablet. The toner is obtained by formulating a pigment suspension through adding purified water into the pigment, then atomizing and spraying the pigment suspension onto dextrin, mixing thoroughly, drying and pulverizing. The application further discloses a lysozyme buccal tablet and a preparation method thereof.

### CROSS-REFERENCE TO RELATED APPLICATION

This application is a divisional of U.S. application Ser. No. 17/319,113, filed on May 13, 2021, which is a continuation of PCT application No. PCT/CN2021/077412, filed on Feb. 23, 2021, which claims the priority and benefit of China Application No. 202010866046.7, filed on Aug. 25, 2020. The entireties of U.S. application Ser. No. 17/319,113, PCT application No. PCT/CN2021/077412, and Chinese application No. 202010866046.7 are hereby incorporated by reference herein and made a part of this specification.

### TECHNICAL FIELD

The invention relates to the technical field of medicine processing, in particular to a toner; the invention also relates to a lysozyme buccal tablet and a preparation method of the lysozyme buccal tablet.

### BACKGROUND ART

According to the Chinese Pharmacopoeia, a tablet refers to a round or shaped tablet-like solid supported by a raw drug and suitable excipients. Most tablets are taken orally, buccally and chewably. In order to improve the willingness of children to take tablets, a certain amount of pigment is often added into the tablets, so that the tablets have bright color and luster.

In the prior art, the pigment is generally directly added into the raw material of the tablet to be granulated together, and the color and luster of the buccal tablet is adjusted through the pigment.

In this process, the pigment is directly dissolved in water, then the pigment and other materials are mixed together, granulated, dried and tableted, and the color and luster of the prepared tablet is adjusted through the pigment. In practical operation, however, it is found that the color and luster inside and outside the tablets prepared by the above method is not uniform, and spots formed by nonuniform color can exist on some tablets, commonly known as “spotted tablet” phenomenon, which affects the uniformity of the tablets.

### SUMMARY

In view of the shortcomings of the prior art, in a first aspect, the present application provides a toner which helps to make the overall color and luster of a tablet uniform during tablet processing and does not easily cause the phenomenon of “spotted tablet”.

In a second aspect, the present application provides a lysozyme buccal tablet in which the pigment is distributed more uniformly so that the overall morphology is more uniform.

In a third aspect, the present application provides a preparation method of the lysozyme buccal tablet according to the second aspect, and the lysozyme buccal tablet prepared by the preparation method has good uniformity, which is beneficial to reducing the phenomenon of “spotted tablet” in the lysozyme buccal tablet, and enables the inner color and the outer color and luster of the lysozyme buccal tablet to be uniform.

In some embodiments, a toner is provided, which is prepared by the following method:
S1, adding a pigment into purified water to prepare a suspension, and uniformly stirring to prepare a pigment suspension;
S2, weighing dextrin, atomizing the pigment suspension obtained in the step S1 and spraying the pigment suspension onto the dextrin under stirring while spraying, adding additional water after spraying the pigment suspension, and continuously stirring until forming to obtain a wet pigment material;
S3, drying and pulverizing the pigment wet material obtained in the step S2 to obtain a toner; wherein the particle size of the pulverized toner is not more than 120 meshes.

In the above technical solution, the dextrin adsorbs the pigment and serves as a support of the pigment, so that the pigment can be uniformly dispersed. Whether the pigment is dissolved in water or not, the pigment can be uniformly dispersed after being adsorbed by dextrin. Then the obtained wet pigment material is dried and pulverized to form the powdery toner. With the toner in the tablet preparation process, after the powder is mixed with the auxiliary materials in the tablet and uniformly stirred, a good mixing effect can be achieved, the color and luster uniformity of the prepared tablet is improved, and the spotted tablet phenomenon generated during tablet processing is reduced.

In some embodiments, in step S2, the system is maintained at an air pressure of 0.5-10 kPa while the pigment suspension is atomized and sprayed onto the dextrin.

In a low-pressure state, the atomized liquid formed by atomization of the pigment suspension can be more uniformly mixed with dextrin, so that the prepared toner has better uniformity, the color and luster of the particles obtained after pulverizing is more uniform, and the possibility of generating spotted tablet in subsequent use is further reduced.

In some embodiments, in step S2, the atomization flow rate is 700-1000 mL/min, and the atomization pressure is 0.1-0.2 mPa.

In some embodiments, the pigment is a mixed system of β-carotene and lemon yellow, and the mass ratio of β-carotene to lemon yellow is (3-6):1; the dextrin is white dextrin, and the mass ratio of the pigment to the dextrin is (0.01-0.05):1.

β-carotene and lemon yellow are mutually coordinated, so that a bright color and luster can be realized. Dextrin is a product obtained by primary hydrolysis of starch, and the surface of dextrin contains more hydroxyl groups, so that the two pigments have good compatibility, the two pigments can be carried uniformly, and the two pigments can be uniformly mixed.

In some embodiments, a lysozyme buccal tablet is provided, including the following raw materials in parts by mass:
filler: 400-525 parts;
sweetener: 10-20 parts;
lysozyme: 15-25 parts;
toner: 20-30 parts
binders: 300-400 parts;
wherein the toner is any one of the above toners; the filler is a mixture of dextrin and sucrose, wherein the dextrin is 130-185 in parts by mass; the sweetener is mannitol, and the binder comprises a 10% povidone K30 solution.

Dextrin has good adsorption property, and has good adsorption property for materials such as lysozyme, toner, sweetener and the like. The sucrose has an adhesion promoting effect, and materials such as dextrin, lysozyme, toner, sweetener and the like can be more fully mixed. In addition, hydrogen bonds are easily formed between the sucrose and the dextrin, so that the sucrose adheres to the surface of the dextrin during processing, and the dextrin is coated, so that the surface of the prepared buccal tablet has sweet taste. The mannitol can be well adsorbed in the dextrin and coated by the dextrin, and the mannitol has high sweetness and refreshing mouthfeel, so that the mouthfeel of the lysozyme buccal tablet can be effectively adjusted. In the process of mixing the toner and the filler, the sucrose and dextrin in the filler can be uniformly mixed with the toner, so that the toner is not easy to agglomerate and block in the mixing process, the dispersion uniformity degree of the toner in the prepared buccal tablet is further improved, the color and luster uniformity degree of the lysozyme buccal tablet is improved, which make lysozyme buccal tablet less likely to be “spotted tablet” due to toner or pigment agglomeration. In addition, the pigment is uniformly distributed in the lysozyme buccal tablet, so that the prepared lysozyme buccal tablet is relatively high in strength, easy to form and not easy to break to form powder or granular.

In some embodiments, the binder further contains 5-12 parts by mass of citric acid.

The citric acid not only can play a role in seasoning to make the mouthfeel of the lysozyme buccal tablet mild and tasty, but also can play a role in resisting oxidation and prolonging the shelf life of the lysozyme buccal tablet.

In some embodiments, a preparation method for preparing the lysozyme buccal tablet is provided, comprising the following steps of:
P1, weighing the filler, the sweetener, the lysozyme, the toner and the citric acid, and sieving for later use;
P2, adding the filler, the sweetener, the lysozyme and the toner into a granulator, and fully stirring to obtain a premixed composition;
P3, adding a binder into the premixed composition obtained in the step P3 in a granulator under stirring to obtain a paste composition;
P4, granulating the paste composition obtained in the step P3 into particles in a granulator to obtain a wet product;
and P5, granulating, drying and tableting the wet product obtained in the step P4 to obtain the lysozyme buccal tablet.

Because both the filler and the toner contain dextrin, the filler and the flavoring agent can form a relatively uniform mixing structure in the mixing process, and the flavoring agent is not easy to agglomerate or block. When the flavoring agent, the sweetener and the lysozyme are fully mixed in the system, a binder is added, the materials are granulated by stirring and cutting, the inner color and the outer color and luster of the lysozyme buccal tablets are uniform after tableting shaping, and the phenomenon of “spotted tablet” is basically avoided.

In some embodiments, the step P2 is specifically as follows: firstly, adding dextrin, lysozyme and toner into a granulator, fully and uniformly stirring, then adding sucrose and mannitol into the granulator, continuously stirring and uniformly mixing to obtain a premixed composition.

The dextrin is used as a main filler, which is the same as the main component in the toner, so that a homogeneous system is easier to form when the dextrin is mixed. After the sucrose and the mannitol are added, the sucrose and the mannitol can be adhered to the dextrin through mutual adsorption of hydroxyl groups to form a coating structure, so that a uniform system is formed, and the prepared lysozyme buccal tablet is more uniform.

In some embodiments, in the step P2, while adding sucrose and mannitol into the granulator, adding purified water of 50-80 parts by mass into the granulator in an atomizing manner; adding sucrose, mannitol and purified water evenly to the granulator over 20-40 s.

Water is simultaneously added in the sucrose adding process, syrup with certain viscosity can be formed, and materials such as dextrin, lysozyme powder, toner and the like formed in the system are preliminarily bonded and shaped through the syrup. Because water is added in the form of atomization, the dextrin can be fully contacted with water during the adding process, which helps the dextrin surface to absorb sucrose more evenly, thus improving the overall strength and uniformity of the formed lysozyme tablet, it can not only improve the color and luster uniformity of lysozyme buccal tablets and reduce the occurrence of “spotted tablet”, but also help to improve the strength of the tablets themselves.

In some embodiments, in the step P3, adding a mixture of 75-150 parts by mass of a 10% povidone-K30 solution and 5-12 parts by mass of citric acid into a granulator within 1-2 min during the addition of the binder, then adding 138-320 parts by mass of a 10% povidone K30 solution, continuously stirring for 10-20 min at a stirring speed of 7-12 r/s to obtain the paste composition.

In the above technical solution, the citric acid and solid materials such as dextrin can be uniformly mixed, and can be coated by the binder. The addition of the binder is then continued to shape the buccal tablet. The paste composition prepared in the process is uniformly mixed, and a stable coating structure can be formed in the cutting process, so that the inner and outer mouthfeel of the prepared lysozyme buccal tablet is uniform.

In summary, the application comprises at least one of the following beneficial technical effects:

1. In the present application, a toner is provided, by preparing the pigment into a pigment suspension and atomizing and spraying into dextrin to prepare powder, the dispersity of the pigment in the preparation of tablets can be improved, the uniformity of color and luster of the tablets after processing is better, and the phenomenon of “spotted tablet” is not easy to generate.

2. In the application, a lysozyme buccal tablet is provided, and the toner is added, so that the lysozyme buccal tablet has better color and luster uniformity.

3. In the present application, a preparation method of the lysozyme buccal tablet is provided, and the prepared lysozyme buccal tablet is uniform in color and luster and less in occurrence of “spotted tablet”.

### DETAILED DESCRIPTION

The technical scheme is further illustrated by specific examples. Those skilled in the art will understand that, these embodiments are provided merely for the purpose of better understanding the present application, not intended to limit the scope of the present application in any way.

Example 1, a toner was prepared by the following steps of:
S1, adding the pigment into purified water to prepare a suspension with the mass fraction of 10%, and uniformly stirring to prepare a pigment suspension;
S2, weighing dextrin, under the normal pressure, atomizing and spraying the pigment suspension obtained in the step S1 onto the dextrin under the pressure of 0.1 mPa and the flow rate of 700 mL/min, stirring the dextrin at the speed of 8 r/s while spraying, adding additional 2.5 g of purified water after the pigment suspension was sprayed, and continuously stirring for 5 min until shaping to obtain a wet pigment material;
and S3, drying the pigment wet material obtained in the step S2 in a hot air circulating oven at a temperature of 75+/−5° C., pulverizing in a pulverizer, and sieving through a 120-mesh sieve to obtain the toner.

In particular, the pigment is a mixed system of 0.4 g β-carotene and 0.1 g lemon yellow, the purified water is added in the pigment suspension in an amount of 5 g, dextrin is white dextrin, and the dextrin is added in an amount of 25 g.

Example 2, a toner prepared by the following steps of:
S1, adding the pigment into purified water to prepare a suspension, and uniformly stirring to prepare a pigment suspension;
S2, weighing dextrin, under the normal pressure, atomizing and spraying the pigment suspension obtained in the step S1 on the dextrin under the pressure of 0.2 mPa and the flow rate of 1000 mL/min, stirring the dextrin at the speed of 8 r/s while spraying, adding additional 2.5 g of purified water after the pigment suspension is sprayed, and continuously stirring for 5 min until shaping to obtain a wet pigment material;
and S3, drying the pigment wet material obtained in the step S2 in a hot air circulating oven at a temperature of 75+/−5° C., pulverizing in a pulverizer, and sieving through a 120-mesh sieve to obtain the toner.

In particular, the pigment is a mixed system of 0.4 g β-carotene and 0.1 g lemon yellow, the purified water is added in the pigment suspension in an amount of 5 g, dextrin is white dextrin, and the dextrin is added in an amount of 25 g.

Example 3: a toner differs from Example 1 in that: the pigment is a mixed system of 0.6 g β-carotene and 0.2 g lemon yellow, and dextrin is added in an amount of 15 g.

Example 4: a toner differs from Example 1 in that: the pigment is a mixed system of 0.45 g β-carotene and 0.075 g lemon yellow, and dextrin is added in an amount of 45.5 g.

Example 5: a toner, which differs from Example 1 in that lemon yellow is replaced with an equivalent amount of β-carotene.

Example 6: a toner that differs from Example 1 in that β-carotene is replaced with an equivalent amount of lemon yellow.

Example 7, a toner prepared by the following steps of:
S1, adding purified water into the pigment to prepare a suspension with the mass fraction of 10%, and uniformly stirring to prepare a pigment suspension;
S2, weighing dextrin, under a atmospheric pressure environment of 0.5 kPa, atomizing and spraying the pigment suspension obtained in the step S1 on the dextrin under the pressure of 0.1 mPa and the flow rate of 700 mL/min, stirring the dextrin at the speed of 8 r/s while spraying, adding additional 2.5 g of purified water after the pigment suspension is sprayed, and continuously stirring for 5 min until shaping to obtain a wet pigment material;
and S3, drying the pigment wet material in the step S2 in a hot air circulating oven at a temperature of 75+/−5° C., pulverizing in a pulverizer, and sieving through a 120-mesh sieve to obtain the toner.

In particular, the pigment is a mixed system of 0.4 g β-carotene and 0.1 g lemon yellow, the purified water is added in the pigment suspension in an amount of 5 g, dextrin is white dextrin, and the dextrin is added in an amount of 25 g.

Example 8, a toner, which differs from Example 7 in that the atmospheric pressure environment of Step S2 is 2 kPa.

Example 9, a toner, which differs from Example 7 in that the atmospheric pressure environment of Step S2 is 6 kPa.

Example 10, a toner, which differs from Example 7 in that the atmospheric pressure environment of Step S2 is 10 kPa.

Comparative Example 1: a toner, which differs from Example 1 in that, in Step S3, after the pigment wet material is dried and pulverized, it is directly used without sieving through a 120-mesh sieve.

Examples 11-20, a lysozyme buccal tablet including the following component materials:
lysozyme: 20 g;
filler: 440 g;
sweetener: 15 g;
toner: 25 g;
binder: 350 g;
among them, the toners in Examples 11-20 are the toners prepared in Examples 1-10, respectively.

In Examples 11-20, the binder is a mixture of, in mass fraction, 342 g of a 10% povidone K30 solution and 8 g of citric acid, the filler is a mixture of 300 g of sucrose and 140 g of dextrin, and the sweetener is mannitol.

The lysozyme buccal tablets of Examples 11-20 are prepared as follows:
P1, weighting the filler, sweetener, lysozyme, toner and citric acid in accordance with the above proportion, weighing and sieving for later use;
P2, adding a filler, a sweetener, a lysozyme and a toner into a granulator, and stirring for 4 min at a speed of 5 r/s to obtain a premixed composition;
P3, adding a binder into the premixed composition obtained in the step P3 in a granulator under the condition of maintaining stirring, stirring for 15 min at the speed of 7 r/s, so that the material is basically in a soft material state to obtain a paste composition;
P4, starting a cutter in the granulator, wherein the rotating speed of the cutter is 25 r/s, cutting the paste composition obtained in the step P3 in the granulator for 60 s to obtain a wet product;
and P5, evenly spreading the wet product obtained in the step P4 on a stainless steel drying plate, feeding a drying frame into an RXH-27-C hot air circulation drying oven, turning on a power switch of the drying oven after the door of the drying oven is closed, simultaneously turning on an automatic temperature control switch on a control panel of the drying oven, adjusting the temperature of the drying oven to be 75° C., and timing drying for 4 hours after the temperature of the drying oven reaches 75° C. Then filling the dried granules into a YK-160 oscillating granulator cavity through a 20-mesh sieve, and starting the YK-160 oscillating granulator to a proper speed. Pour the dried granulation into the hopper of the YK-160 oscillating granulator, slowly turn on the feed switch and control the slower feed rate. And after finishing the granulation, tableting the obtained granules to complete the lysozyme buccal tablet.

Example 21, a lysozyme buccal tablet, which differs from Example 11 in that it comprises the following component materials:
lysozyme: 15 g;
filler: 400 g;
sweetener: 10 g;
toner: 20 g;
binder: 400 g;
wherein, the filler is a binder which is a mixture of, in mass fraction, 395 g of a 10% povidone K30 solution and 5 g of citric acid, the filler is a mixture of 270 g of sucrose and 130 g of dextrin, and the sweetener is mannitol.

Example 22, a lysozyme buccal tablet, which differs from Example 11 in that it comprises the following component materials:
lysozyme: 25 g;
filler: 525 g;
sweetener: 20 g;
toner: 30 g;
binder: 300 g;
wherein, the filler is a binder which is a mixture of, in mass fraction, 288 g of a 10% povidone K30 solution and 12 g of citric acid, the filler is a mixture of 270 g of sucrose and 130 g of dextrin, and the sweetener is mannitol.

Example 23, a lysozyme buccal tablet, which differs from Example 17 in comprising the following component materials:
lysozyme: 15 g;
filler: 400 g;
sweetener: 10 g;
toner: 20 g;
binder: 400 g;
wherein, the filler is a binder which is a mixture of, in mass fraction, 395 g of a 10% povidone K30 solution and 5 g of citric acid, the filler is a mixture of 270 g of sucrose and 130 g of dextrin, and the sweetener is mannitol.

Example 24, a lysozyme buccal tablet, which differs from Example 17 in that it comprises the following component materials:
lysozyme: 25 g;
filler: 525 g;
sweetener: 20 g;
toner: 30 g;
binder: 300 g;
wherein, the filler is a binder which is a mixture of, in mass fraction, 288 g of a 10% povidone K30 solution and 12 g of citric acid, the filler is a mixture of 270 g of sucrose and 130 g of dextrin, and the sweetener is mannitol.

Example 25, a lysozyme buccal tablet, which differs from Example 17 in that the binder is, in mass fraction, 350 g of a 10% of povidone K30 solution.

Example 26, a lysozyme buccal tablet, which differs from Example 17 in that Step P2 is specifically as follows: firstly, adding dextrin, lysozyme and toner into a granulator, stirring for 10 min at the rotation speed of 7 r/s, then adding sucrose and mannitol into the granulator, and continuously stirring for 10 min to obtain the premixed composition.

Example 27, a lysozyme buccal tablet, which differs from Example 26 in that Step P2 is specifically as follows: firstly, adding dextrin, lysozyme and toner into a granulator, stirring for 5 min at the rotation speed of 7 r/s, then adding sucrose and mannitol into the granulator, and continuously stirring for 10 min to obtain the premixed composition.

Example 28, a lysozyme buccal tablet, which differs from Example 26 in that Step P2 is specifically as follows: firstly, adding dextrin and lysozyme into a granulator, stirring for 10 min at the rotation speed of 7 r/s, then adding sucrose, mannitol and toner into the granulator, and continuously stirring for 10 min to obtain the premixed composition.

Example 29, a lysozyme buccal tablet, which differs from Example 26 in that Step P2 is specifically as follows: firstly, adding dextrin and sucrose into a granulator, stirring for 10 min at the rotation speed of 7 r/s, then adding lysozyme, mannitol and toner into the granulator, and continuously stirring for 10 min to obtain a premixed composition.

Example 30, a lysozyme buccal tablet, which differs from Example 26 in that Step P2 is specifically as follows: firstly adding dextrin, lysozyme and toner into a granulator, stirring for 10 min at the rotation speed of 7 r/s, then adding sucrose and mannitol into the granulator, spraying 50 g of water into the granulator in an atomization mode, uniformly adding the sucrose, the mannitol and the water within 20 s under the atomization pressure of 0.1 kPa, and continuously stirring for 10 min after finishing the addition to obtain the premixed composition.

Example 31, a lysozyme buccal tablet, which differs from Example 26 in that Step P2 is specifically as follows: firstly adding dextrin, lysozyme and toner into a granulator, stirring for 10 min at the rotation speed of 7 r/s, then adding sucrose and mannitol into the granulator, spraying 80 g of water into the granulator in an atomization mode, uniformly adding the sucrose, the mannitol and the water within 40 s under the atomization pressure of 0.1 kPa, and continuously stirring for 10 min after finishing the addition to obtain the premixed composition.

Example 32, a lysozyme buccal tablet, which differs from Example 26 in that Step P2 is specifically as follows: firstly, adding dextrin, lysozyme and toner into a granulator, stirring at the rotation speed of 7 r/s for 10 min, then adding sucrose and mannitol into the granulator, and pouring 50 g of water into the granulator, continuously stirring for 10 min after finishing the addition to obtain the premixed composition.

Example 33. A lysozyme buccal tablet, which differs from Example 31 in that in Step P3, 75 parts of a 10% povidone-K30 solution and 5 parts of citric acid are mixed and uniformly added to a granulator within 1 min during the addition of the binder, and then 215 parts by mass of a 10% povidone-K30 solution are added to the granulator to stir at 7 r/s for 20 min.

Example 34. A lysozyme buccal tablet, which differs from Example 33 in that in Step P3, 150 parts of a 10% povidone-K30 solution and 12 parts of citric acid are mixed and uniformly added to a granulator within 1 min during the addition of the binder, and then 138 parts by mass of a 10% povidone-K30 solution are added to the granulator to stir at 12 r/s for 10 min.

Example 35, a lysozyme buccal tablet, which differs from Example 11 in that it comprises the following component materials:
lysozyme: 25 g;
filler: 525 g;
sweetener: 20 g;
toner: 30 g.

Example 35 Tablets are prepared by mixing the above ingredients, dosing, tabletting and shaping.

The lysozyme buccal tablets prepared in Examples 11-35 are compared by setting the following comparative examples.

Comparative Example 2, a lysozyme buccal tablet, which differs from Example 11 in that the toner prepared in Comparative Example 1 is selected as the toner.

Comparative Example 3, a lysozyme buccal tablet, which differs from Example 11 in that it comprises the following component materials:
lysozyme: 20 g;
filler: 440 g;
sweetener: 15 g;
toner: 25 g;
binder: 350 g;
wherein the toner is the toner prepared in Example 1, the binder is a mixture of, in mass fraction, 342 g of a 10% povidone K30 solution and 8 g of citric acid, the filler is 440 g of sucrose, and the sweetener is mannitol.

Comparative Example 4, a lysozyme buccal tablet comprising the following component materials:
lysozyme: 20 g;
filler: 440 g;
sweetener: 15 g;
toner: 25 g;
binder: 350 g;
wherein, the toner is the toner prepared in Example 1, the binder is a mixture of, in mass fraction, 342 g of a 10% povidone K30 solution and 8 g of citric acid, the filler is 440 g of dextrin, and the sweetener is mannitol.

Comparative Example 5, a lysozyme buccal tablet, the preparation process thereof comprises the following steps of:

1. adding the filler, the sweetener, the lysozyme and the pigment into a granulator, fully stirring and uniformly mixing; wherein the filler is a composition formed by mixing 165 g of dextrin and 300 g of sucrose, the pigment is a composition formed by mixing 0.4 g of β-carotene and 0.1 g of lemon yellow, and the sweetener is mannitol.

2. adding a binder into the composition obtained in the step 1, and stirring for 10 min at the speed of 7 r/s, so that the material is basically in a soft material state; wherein, the binder is a mixture of, in mass fraction, 342 g of a 10% povidone K30 solution and 8 g of citric acid.

3. starting a cutter in the granulator, wherein the rotating speed of the cutter is 25 r/s, cutting the material obtained in the step 2 into particles for 60 s.

4. evenly spreading the wet product obtained in the step 3 on a stainless steel drying plate, feeding into a hot air circulation drying oven, adjusting the temperature of the drying oven to be 75° C., and timing drying for 4 hours; then passing the dried granules through a 20-mesh sieve, filling into an oscillating granulator cavity, and sizing the particles at a slow feed rate. And after finishing the granulation, tableting the obtained granules to complete the lysozyme buccal tablet.

For Examples 11-35 and Comparative Examples 2-5, the following experiments are set up to determine the properties of each aspect.

Experiment 1. Appearance standard, 100 samples of each group of examples and comparative examples are inspected by visual inspection, and the inspection standard is as follows: the lysozyme has consistent tablet shape, smooth tablet surface, tidy edge, uniform surface and internal color and luster.

Experiment 2. Fragility test, the friability of the lysozyme buccal tablets is tested according to the method of 0923 tablet friability test in Chinese Pharmacopoeia 2015. The specific detection method comprises the following steps of: taking 10 tablets of lysozyme buccal tablet, blowing off the surface blown-off powder by a blower, weighing, placing in a CS-2 tablet friability tester, setting the rotation speed to be 25 r/min, taking out after 100 times of rotation, blowing off the powder, weighing, and measuring weight loss.

Experiments 1 and 2 are carried out for Examples 11-16 and Comparative Examples 2-5, and the experimental results obtained are shown in Table 1.

TABLE 1
characterization of Related Traits for Examples 11-16 and Comparative Examples 2-5

| | | | Number of | |
|---|---|---|---|---|
| Sequence | Surface color and luster | Internal color and luster | tablets of uneven | Weight loss |
| Number | uniformity | uniformity | color and luster | ratio (%) |
| Example 11 | A small number of tablets | With a certain amount of | 13 | 0.20 |
| | have uneven color and luster | spots inside | | |
| | and spots on the surface | | | |
| Example 12 | A small number of tablets | With a certain amount of | 12 | 0.22 |
| | have uneven color and luster | spots inside | | |
| | and spots on the surface | | | |
| Example 13 | A small number of tablets | With a certain amount of | 13 | 0.23 |
| | have uneven color and luster | spots inside | | |
| | and spots on the surface | | | |
| Example 14 | A small number of tablets | With a certain amount of | 15 | 0.25 |
| | have uneven color and luster | spots inside | | |
| | and spots on the surface | | | |
| Example 15 | A small number of tablets | With a certain amount of | 13 | 0.19 |
| | have uneven color and luster | spots inside | | |
| | and spots on the surface | | | |
| Example 16 | A small number of tablets | With a certain amount of | 15 | 0.23 |
| | have uneven color and luster | spots inside | | |
| | and spots on the surface | | | |
| Comparative | With spots on most tablets | Internal color consistent | 82 | 3.98 |
| Example 2 | | with the surface color, | | |
| | | more spots | | |
| Comparative | With spots on the tablets, | Internal color almost | 45 | 0.77 |
| Example 3 | “spotted tablet” phenomenon | consistent with the | | |
| | is serious | surface color, with spots | | |
| Comparative | Uneven color, with “spotted | Internal color almost | 27 | 1.42 |
| Example 4 | tablet” phenomenon | consistent with the | | |
| | | surface color, some of | | |
| | | the tablets have spots | | |
| Comparative | The color uniformity is poor, | Internal color almost | 48 | 0.41 |
| Example 5 | with spots and “spotted | consistent with the | | |
| | tablet” phenomenon | surface color, more | | |
| | | spots | | |

According to the experimental data, in the technical solutions of Examples 11-16, the pigment and the dextrin are firstly prepared into the toner, so that the toner and the filler are more uniformly mixed in the subsequent lysozyme buccal tablet production process, and the color and luster uniformity of the prepared lysozyme buccal tablet is improved. In Comparative Example 2, since the toner is not sieved after the processing is completed, a small amount of agglomerated toner is mixed in the lysozyme buccal tablet during the subsequent processing, resulting in an increase in the friability of the lysozyme buccal tablet and a tendency to appear spots.

In the Comparative Example 3, the filler is only sucrose in the lysozyme preparation process, so the effect on the toner is poor, resulting in the phenomenon that the lysozyme buccal tablets produced contain more unevenness. In the Comparative Example 4, the filler is only dextrin, and the lack of the adhesion promotion and dispersion effect of sucrose can lead to uneven dispersion of the toner, sweetener, lysozyme and other materials in the dextrin during the mixing process of the toner and the filler, so that partial uneven spots remain on the surface of the prepared lysozyme buccal tablet. And the lack of the adhesion promoting effect of sucrose can lead to the reduction of the strength of lysozyme and the increase of the friability.

In Comparative Example 5, the pigment is directly mixed with other raw materials and lysozyme buccal tablets are prepared. In the above process, the distribution uniformity of the pigment in the lysozyme buccal tablets is poor, the pigment itself is easy to agglomerate, and it is difficult to uniformly distribute the pigment into the whole system only by stirring. Therefore, compared with Examples 11-16, the lysozyme buccal tablets prepared in Comparative Example 5 had poor uniformity.

Experiments 1 and 2 are carried out for the lysozyme buccal tablets prepared in Example 11 and Examples 17-25, and the results are shown in Table 2.

TABLE 2
characterization of Related Traits for Example 11 and Examples 17-25.

| | | | Number of | |
|---|---|---|---|---|
| Sequence | Surface color and luster | Internal color and luster | tablets of uneven | Weight loss |
| Number | uniformity | uniformity | color and luster | ratio (%) |
| Example 11 | Part of the tablets have | With a certain amount of | 13 | 0.20 |
| | uneven color and luster | spots inside | | |
| | and spots on the surface | | | |
| Example 17 | Part of the tablets have | The inner color of the | 9 | 0.24 |
| | uneven color and luster | tablet is close to the | | |
| | on the surface | surface, and some tablets | | |
| | | are not uniform | | |
| Example 18 | Part of the tablets have | The inner color of the | 8 | 0.18 |
| | uneven color and luster | tablet is close to the | | |
| | on the surface | surface, and some tablets | | |
| | | are not uniform | | |
| Example 19 | Part of the tablets have | The inner color of the | 9 | 0.19 |
| | uneven color and luster | tablet is close to the | | |
| | on the surface | surface, and some tablets | | |
| | | are not uniform | | |
| Example 20 | Part of the tablets have | The inner color of the | 10 | 0.21 |
| | uneven color and luster | tablet is close to the | | |
| | on the surface | surface, and some tablets | | |
| | | are not uniform | | |
| Example 21 | Part of the tablets have | With a certain amount of | 14 | 0.20 |
| | uneven color and luster | spots inside | | |
| | and spots on the surface | | | |
| Example 22 | Part of the tablets have | With a certain amount of | 13 | 0.20 |
| | uneven color and luster | spots inside | | |
| | and spots on the surface | | | |
| Example 23 | Part of the tablets have | The inner color of the | 8 | 0.19 |
| | uneven color and luster | tablet is close to the | | |
| | on the surface | surface, and some tablets | | |
| | | are not uniform | | |
| Example 24 | Part of the tablets have | The inner color of the | 10 | 0.22 |
| | uneven color and luster | tablet is close to the | | |
| | on the surface | surface, and some tablets | | |
| | | are not uniform | | |
| Example 25 | Part of the tablets have | The inner color of the | 9 | 0.21 |
| | uneven color and luster | tablet is close to the | | |
| | on the surface | surface, and some tablets | | |
| | | are not uniform | | |

In the above examples, in the process of preparing the toner in Example 11, Example 21, and Example 22, step S2 is carried out at atmospheric pressure. While in the process of preparing the toner in Examples 17-20 and Examples 23 and 24, Step S2 is carried out at a low pressure. By comparing the above data, it can be seen that the atomization spray operation of the pigment suspension at low pressure helps to improve the uniformity of the prepared lysozyme buccal tablet. It is presumed that the principle may be that under the condition of low pressure, during the atomization process of the pigment suspension, the liquid droplets and the liquid droplets are not easy to agglomerate, so that the atomized small liquid droplets are more uniformly dispersed in dextrin, and the uniformity of the prepared toner is improved.

In example 25, citric acid is not contained in the binder, the taste is poor, and the shelf life of the prepared lysozyme buccal tablet is affected.

Experiments 1 and 2 are carried out for the lysozyme buccal tablets prepared in Example 17 and Examples 26-35, and the results are shown in Table 3.

TABLE 3
characterization of Related Traits for Example 17 and Examples 26-35

| | | | Number of | |
|---|---|---|---|---|
| Sequence | Surface color and luster | Internal color and luster | tablets of uneven | Weight loss |
| Number | uniformity | uniformity | color and luster | ratio (%) |
| Example 17 | Part of the tablets have | The inner color of the tablet | 9 | 0.24 |
| | uneven color and luster | is close to the surface, and | | |
| | on the surface | some tablets are not | | |
| | | uniform | | |
| Example 26 | A small number of tablets | The inner color of the tablet | 5 | 0.21 |
| | have uneven color and | is close to the surface, and | | |
| | luster on the surface | some tablets are not | | |
| | | uniform | | |
| Example 27 | A small number of tablets | The inner color of the tablet | 4 | 0.19 |
| | have uneven color and | is close to the surface, and | | |
| | luster on the surface | some tablets are not | | |
| | | uniform | | |
| Example 28 | A small number of tablets | The interior is lighter in | 10 | 0.21 |
| | have uneven color and | color than the surface and | | |
| | luster on the surface | some of the tablets are not | | |
| | | uniform | | |
| Example 29 | Part of the tablet is | The interior is lighter in | 16 | 0.24 |
| | uneven on the surface | color than the surface and | | |
| | | almost uniform | | |
| Example 30 | The color and luster are | The color of the interior is | 0 | <0.1 |
| | uniform, and the | consistent with that of the | | |
| | phenomenon of spots and | surface, and the | | |
| | spotted tablet is avoided | phenomenon of spots and | | |
| | | spotted tablet is avoided | | |
| Example 31 | The color and luster are | The color of the interior is | 1 | <0.1 |
| | uniform, and the | consistent with that of the | | |
| | phenomenon of spots and | surface, and the | | |
| | spotted tablet is avoided | phenomenon of spots and | | |
| | | spotted tablet is avoided | | |
| Example 32 | A small number of tablets | The inner color is darker | 8 | <0.1 |
| | have uneven color and | than the outer, with a small | | |
| | luster on the surface | amount of unevenness in | | |
| | | some tablets | | |
| Example 33 | The color and luster are | The color of the interior is | 0 | <0.1 |
| | uniform, and the | consistent with that of the | | |
| | phenomenon of spots and | surface, and the | | |
| | spotted tablet is avoided | phenomenon of spots and | | |
| | | spotted tablet is avoided | | |
| Example 34 | The color and luster are | The color of the interior is | 0 | <0.1 |
| | uniform, and the | consistent with that of the | | |
| | phenomenon of spots and | surface, and the | | |
| | spotted tablet is avoided | phenomenon of spots and | | |
| | | spotted tablet is avoided | | |
| Example 35 | A small number of tablets | The inner color of the tablet | 11 | 0.36 |
| | have uneven color and | is close to the surface, and | | |
| | luster and spots on the | some tablets are not | | |
| | surface | uniform | | |

The order of addition of the materials is adjusted in Examples 26-29. As can be seen from the data in Table 4, the color and luster uniformity of the prepared lysozyme buccal tablets (as shown in Examples 26 and 27) can be improved by the method of adding dextrin, lysozyme and toner to a granulator for mixing and then adding sucrose and mannitol. In the embodiment 28, firstly, dextrin and lysozyme are mixed, then sucrose, a toner and a sweetener are added, and the toner is not uniformly dispersed in the sucrose, so that the color and luster uniformity is poor. In Example 29, dextrin and sucrose are mixed before lysozyme, mannitol and toner are added. Due to the mutual attraction effect between the dextrin and the sucrose, after the dextrin and the sucrose are mixed, other materials are easy to agglomerate when added, so that the color and luster uniformity of the prepared lysozyme buccal tablet is poor.

In Examples 30-31, purified water is sprayed into the system in an atomized manner while sucrose is added. When the purified water is sprayed, part of sucrose can be dissolved by the purified water to form syrup, and the mutual adsorption effect of dextrin and sucrose is enhanced, so that the system is changed from powder to paste with slight viscosity, at the moment, materials such as pigment, mannitol and the like added into the granulator can be positioned by surrounding dextrin in the process of adding into the granulator, and the materials are not easy to agglomerate due to mutual adsorption in the stirring process. In the embodiment 32, all the water is directly added, so that dextrin is quickly agglomerated to form lumps in the processing process, the subsequent stirring effect is influenced, and the lysozyme buccal tablet is uneven in texture and uneven in color and luster.

In Examples 33 and 34, further during the addition of the binder, a portion of a mixture of 10% povidone-K30 solution and citric acid is added, and the remaining 10% povidone-K30 solution is added to the system to help uniformly coat the citric acid so that the citric acid could be more uniformly distributed inside and on the surface of the buccal tablet, therefore, the whole taste of the buccal tablet is relatively uniform. In Example 35, tablets are compressed by tableting without the addition of a binder, which is simpler in process.

The examples of the present embodiments are all preferred examples of the present application and are not intended to limit the scope of the present application. Therefore, all equivalent changes made in accordance with the principles of the present application shall be comprised in the scope of protection of the present application.

What is claimed is:

1. A preparation method for preparing a lysozyme buccal tablet, wherein the lysozyme buccal tablet comprises following raw materials in parts by mass:
filler: 400-525 parts;
sweetener: 10-20 parts;
lysozyme: 15-25 parts;
toner: 20-30 parts; and
binder: 300-400 parts;
wherein the filler is a mixture of dextrin and sucrose, and the dextrin is 130-185 in parts by mass; the sweetener is mannitol; the binder comprises a 10% povidone K30 solution; the toner is prepared by following method:
S1, adding a pigment into purified water to prepare a suspension, and uniformly stirring to prepare a pigment suspension;
S2, weighing the dextrin, atomizing the pigment suspension obtained in step S1 and spraying the pigment suspension onto the dextrin under stirring, adding additional water after the pigment suspension spraying, and continuously stirring until shaping to obtain a wet pigment material; and
S3, drying and pulverizing the wet pigment material in step S2 to obtain the toner; wherein a particle size of the pulverized toner is not more than 120 meshes;
the preparation method comprises following steps of:
P1, weighing the filler, the sweetener, the lysozyme, the toner and citric acid, and sieving for later use;
P2, adding the dextrin, the lysozyme and the toner into a granulator under fully stirring, then adding the sucrose and the mannitol into the granulator, continuously stirring and uniformly mixing to obtain a premixed composition;
P3, adding the binder into the premixed composition obtained in step P2 in the granulator under stirring to obtain a paste composition;
P4, cutting the paste composition obtained in step P3 into particles in the granulator to obtain a wet product; and
P5, granulating, drying and tableting the wet product obtained in step P4 to obtain the lysozyme buccal tablet;
wherein, in step P2, when adding the sucrose and the mannitol into the granulator, purified water of 50-80 parts by mass is added into the granulator in an atomizing manner; and the sucrose, the mannitol and the purified water are evenly added to the granulator over 20-40 s.

2. The preparation method of the lysozyme buccal tablet of claim 1, wherein the binder further comprises the citric acid in an amount of 5 to 12 parts by mass.

3. The preparation method of the lysozyme buccal tablet of claim 1, wherein, in step P3, a mixture of 75-150 parts by mass of the 10% povidone K30 solution and 5-12 parts by mass of the citric acid are added into the granulator within 1-2 min during adding the binder, and 138-320 parts by mass of the 10% povidone K30 solution is added, and stirring is performed continuously for 10-20 min at a stirring speed of 7-12 r/s to obtain the paste composition.

4. The preparation method of the lysozyme buccal tablet of claim 1, wherein, in step S2, a flow rate of atomization is 700-1000 mL/min, and an atomization pressure is 0.1-0.2 mPa.

5. The preparation method of the lysozyme buccal tablet of claim 1, wherein the pigment is a mixed system of β-carotene and lemon yellow, and a mass ratio of the β-carotene to the lemon yellow is (3-6):1; the dextrin is white dextrin, and a mass ratio of the pigment to the dextrin is (0.01-0.05):1.
